(19) United States
(12) Patent Application Publication
(10) Pub. No.: US 2024/0252309 A1
(43) Pub. Date: Aug. 1, 2024

(54) DOUBLE-SIDED ASPHERIC DIFFRACTIVE MULTIFOCAL LENS, MANUFACTURE, AND USES THEREOF

(71) Applicant: AST Products, Inc., Billerica, MA (US)

(72) Inventors: Yi-Feng Chiu, Hsinchu (TW); Chuan-Hui Yang, Taichung (TW); Wen-Chu Tseng, Westford, MA (US)

(73) Assignee: AST Products, Inc., Billerica, MA (US)

(21) Appl. No.: 18/636,110

(22) Filed: Apr. 15, 2024

Related U.S. Application Data
(63) Continuation of application No. 17/332,304, filed on May 27, 2021, now Pat. No. 11,963,868.
(60) Provisional application No. 63/032,892, filed on Jun. 1, 2020.

(51) Int. Cl. A61F 2/16

(52) U.S. Cl. CPC A61F 2/164; A61F 2/1618; A61F 2/1654; A61F 2002/1683; A61F 2240/001

(57) ABSTRACT

A double-sided aspheric diffractive multifocal lens and methods of manufacturing and design of such lenses in the field of ophthalmology. The lens can include an optic comprising an aspheric anterior surface and an aspheric posterior surface. On one of the two surfaces a plurality of concentric diffractive multifocal zones can be designed. The other surface can include a toric component. The double-sided aspheric surface design results in improvement of the modulation transfer function (MTF) of the lens-eye combination by aberration reduction and vision contrast enhancement as compared to one-sided aspheric lens. The surface having a plurality of concentric diffractive multifocal zones produces a near focus, an intermediate focus, and a distance focus.

### CROSS-REFERENCE TO RELATED APPLICATIONS

This application is a continuation of U.S. patent application Ser. No. 17/332,304 filed May 27, 2021, which claims priority to U.S. Provisional Application No. 63/032,892 filed on Jun. 1, 2020, the entire contents of each of which are incorporated herein by reference.

### FIELD

The present disclosure relates generally to ophthalmic lenses, and more specifically to a novel double-sided aspheric diffractive multifocal lens, design, manufacture, and uses thereof.

### BACKGROUND

Ophthalmology is the field of medicine directed to the anatomy, physiology and diseases of the human eye. The anatomy of the human eye is rather complex. The main structures of the eye include the cornea, a spherical clear tissue at the outer front of the eye; the iris, which is the colored part of the eye; the pupil, an adaptable aperture in the iris that regulates the amount of light received in the eye; the crystalline lens, a small clear disk inside the eye that focuses light rays onto the retina; the retina is a layer that forms the rear or backside of the eye and transforms sensed light into electrical impulses that travel through the optic nerve to the brain. The posterior chamber, i.e., the space between the retina and the lens, is filled with aqueous humour, and the anterior chamber, i.e., the space between the lens and the cornea, is filled with vitreous humour a clear, jelly-like substance.

The natural crystalline lens has a flexible, transparent, biconvex structure, and together with the cornea, operates to refract light to be focused on the retina. The lens is flatter on its anterior side than on its posterior side and its curvature is controlled by the ciliary muscles to which the lens connects by suspensory ligaments, called zonules. By changing the curvature of the lens, the focal distance of the eye is changed so as to focus on objects at various distances. To view an object at a short distance of the eye, the ciliary muscles contract, and the lens thickens, resulting in a rounder shape and thus high refractive power. Changing focus to an object at a greater distance requires the relaxation of the lens and thus increasing the focal distance. This process of changing curvature and adapting the focal distance of the eye to form a sharp image of an object at the retina is called accommodation.

In humans, the refractive power of the crystalline lens in its natural environment is approximately 18-20 diopters, roughly one-third of the total optical power of the eye. The cornea provides the remaining 40 diopters of the total optical power of the eye.

With the ageing of the eye, the opaqueness of the lens diminishes, called cataract. Some diseases like diabetes, trauma, but also some medications, and excessive UV light exposure may also cause cataract. A cataract is painless and results in a cloudy, blurry vision. Treatments for cataracts include surgery, by which the cloudy lens is removed and replaced with an artificial one, generally called an intraocular lens (IOL or IOLs).

Another age-related effect is called presbyopia, which is manifested by difficulty in reading small print or seeing nearby pictures clearly. Presbyopia generally is believed to be caused by a thickening and loss of flexibility of the natural lens inside the eye. Age-related changes also take place in the ciliary muscles surrounding the lens. With less elasticity it becomes harder to focus at objects close to the eye.

A variety of intraocular lenses are also employed for correcting other visual disorders, such as myopia or nearsightedness, when the eye is unable to see distant objects caused by the cornea having too much curvature, for example. The effect of myopia is that distant light rays focus at a point in front of the retina, rather than directly on its surface. Hyperopia or farsightedness, caused by an abnormally flat cornea, such that light rays entering the eye focus behind the retina, not allowing to focus on objects that are close, and astigmatism, which is another common cause of visual difficulty in which images are blurred due to an irregularly shaped cornea.

In the majority of cases, intraocular lenses are implanted in a patient's eye during cataract surgery, to replace the natural crystalline lens and compensate for the loss of optical power of the removed lens. Modern IOL optics are designed to have a multifocal optic for providing short, intermediary and distance vision of objects, also called multifocal IOL, or more specific trifocal lenses. Presbyopia is corrected by eyeglasses or contact lenses and patient's may also opt for multifocal optics. In some cases, an IOL can include diffractive structures to have not only a far-focus power but also a near-focus power, thereby providing a degree of pseudo-accommodation. However, a variety of aberrations, such as spherical and astigmatic aberrations, can adversely affect the optical performance of such lenses. For example, spherical aberrations can degrade vision contrast, especially for large pupil sizes.

Accordingly, what is needed is intraocular lenses that can simultaneously provide a near focus, an intermediate focus, and a distance focus, which can also address adverse effects such as spherical and astigmatic aberrations, thereby providing enhanced contrast and improved visual acuity.

### SUMMARY

The present disclosure is related to a double-sided aspheric diffractive multifocal lens, which can eliminate spherical and astigmatic aberrations and provide enhanced contrast and improved visual acuity. In some embodiments, the diffractive multifocal lens can include a lens body, the lens body can include: a first aspheric surface; and a second aspheric surface including a central zone and a plurality of diffractive elements comprising concentric annular zones extending in a radial direction, each concentric annular zone having a periodically structured curve comprising two smooth turning points between two sharp turning points, thereby producing a near focus (f₂), an intermediate focus (f₁), and a distance focus (f₀).

In some embodiments, the first aspheric surface is anterior surface, and the second aspheric surface is posterior surface. In some embodiments, the first aspheric surface can include a toric component. In some embodiments, a height profile of the first aspheric surface and/or the second aspheric surface is represented by:

$${Z}_{asp} ( r ) = \frac{c {r}^{2}}{1 + \sqrt{1 - ( 1 + k ) {c}^{2} {r}^{2}}} + {\sum }_{i = 2}^{n} {A}_{i} {r}^{2 i}$$

wherein Zₐₛₚ is the height profile of the aspheric structure, r is the radial distance in millimeters, c is the curvature, k is the conic constant, and Aᵢ is high order aspheric coefficients.

In some embodiments, a height profile of the diffractive elements is represented by:

$${Z}_{diff} ( r ) = {Φ}_{( n )} ( r ) \times \frac{λ}{{n}_{1} - {n}_{0}}$$

wherein λ is the design wavelength, Φ₍ₙ₎(r) is phase profile, n₁ is refractive index of lens material, and n₀ is refractive index of a medium covering the lens.

In some embodiments, phase profile ON (r) can be represented as:

$${Φ}_{( n )} ( r ) = A \times sin ( ( B \times \frac{r - {r}_{n}}{{r}_{n + 1} - {r}_{n}} + C ) \times π ) + D$$

wherein r is the radial distance of the lens in millimeter, rₙ is radius of n^{th} zone, rₙ₊₁ is radius of (n+1)^{th} zone, and A, B, C and D, are light distribution parameters. A is amplitude; B is the period as

$$\frac{2 π}{B} ;$$

C is phase shift; D is vertical shift.

In some embodiments, phase profile Φ₍ₙ₎(r) can be in the range of −4π≤Φ₍ₙ₎(r)≤4π. In some embodiments, the distance focus (f₀), the intermediate focus (f₁), and the near focus (f₂) are in the range of:

$$0 D \leq \frac{1}{{f}_{0}} \leq 55 D , 1 D \leq \frac{1}{{f}_{1}} - \frac{1}{{f}_{0}} \leq 2.5 D , 2 D \leq \frac{1}{{f}_{2}} - \frac{1}{{f}_{0}} \leq 5 D .$$

In some embodiments, the diffractive multifocal lens can be an intraocular lens (IOL). In some embodiments, the diffractive multifocal lens can further include a pair of haptics extended outwardly from the lens body. In some embodiments, the IOL is a posterior chamber IOL, and the posterior chamber IOL is configured to be implanted into capsular bag of a human eye.

In some embodiments, the present disclosure is directed to a method of treating an ophthalmic disease or disorder in a subject, the method can include implanting into an eye of the subject a diffractive multifocal lens comprising a lens body, the lens body can include a first aspheric surface; and a second aspheric surface comprising a central zone and a plurality of diffractive elements comprising concentric annular zones extending in a radial direction, each concentric annular zone having a periodically structured curve comprising two smooth turning points between two sharp turning points.

In some embodiments, the present disclosure is directed to a method of manufacturing a diffractive multifocal lens, the method can include (a) manufacturing a first aspheric surface optionally comprising a toric component; (b) manufacturing a second aspheric surface; and (c) generating a central zone and diffractive elements comprising a plurality of concentric annular zones on the second aspheric surface, each concentric annular zone having a periodically structured curve comprising two smooth turning points between two sharp turning points, thereby producing a near focus (f₂), an intermediate focus (f₁), and a distance focus (f₀). In some embodiment, the method can further include performing an in situ image quality analysis to ensure the performance meets the pre-established quality criteria.

### BRIEF DESCRIPTION OF THE DRAWINGS

FIG. 1A illustrates the top view of a double-sided aspheric multifocal diffractive IOL, according to some embodiments of the present disclosure;

FIG. 1B shows the cross-sectional view of a double-sided aspheric multifocal diffractive IOL, according to some embodiments of the present disclosure;

FIG. 2A illustrates a blow-up view of the lens body of the IOL, according to some embodiments of the present disclosure;

FIG. 2B illustrates the height profile of the diffractive elements, according to some embodiments of the present disclosure;

FIG. 2C illustrates the height profile of the diffractive elements, according to another embodiment of the present disclosure;

FIG. 3A illustrates the optical performance (modulation transfer function, MTF) at a 3 mm aperture and at a resolution measurement of 50 LP/mm by varying parameters according to a first embodiment of the present disclosure;

FIG. 3B illustrates the height profile of the aspheric and diffractive combination structure according to a first embodiment of the present disclosure;

FIG. 4A illustrates the optical performance (MTF) at a 3 mm aperture and at a resolution measurement of 50 LP/mm by varying parameters according to a second embodiment of the present disclosure;

FIG. 4B illustrates the height profile of the aspheric and diffractive combination structure according to a second embodiment of the present disclosure;

FIG. 5A illustrates the optical performance (MTF) at a 3 mm aperture and at a resolution measurement of 50 LP/mm by varying parameters according to a third embodiment of the present disclosure;

FIG. 5B illustrates the height profile of the aspheric and diffractive combination structure according to a third embodiment of the present disclosure; and

FIG. 6 is a flowchart illustrating the design and manufacture of the double-sided aspheric multifocal diffractive IOL, according to some embodiments of the present disclosure.

### DETAILED DESCRIPTION

The present disclosure is related to a double-sided aspheric diffractive multifocal lens and methods of designing and manufacturing of such lenses in the field of ophthalmology. The lens can include an aspheric anterior surface and an aspheric posterior surface. One of the two surfaces can include a plurality of concentric diffractive multifocal zones. The other surface can optionally include a toric component. The double-sided aspheric surface design results in an improvement of the modulation transfer function (MTF) of the lens-eye combination by aberration reduction and vision contrast enhancement as compared to one-sided aspheric lens. The surface having a plurality of concentric diffractive multifocal zones can produce a near focus, an intermediate focus, and a distance focus.

Multifocal IOLs are commonly used to treat presbyopia, a condition in which the eye exhibits a progressively diminished ability to focus on near objects. Human beings become presbyopic due to aging, and the effect typically becomes noticeable starting at about the age of 40-45 years old, when they discover they need reading glasses. Presbyopic individuals who wear corrective lenses may then find that they need two separate prescriptions, preferably within the same bifocal lens, one for reading (near) and another for driving (distance). A trifocal lens can further improve vision at intermediate distances, for example, when working at a computer.

Diffractive IOLs can have a repeating structure that may be formed in the surface of an optical element by a fabrication method such as, for example, cutting the surface using a lathe that may be equipped with a cutting head made of a hard mineral such as diamond or sapphire; direct write patterning using a high energy beam such as a laser beam or electron beam or a similar method of ablating the surface; etching the surface using a photolithographic patterning process; or molding the surface. The diffractive structure is typically a series of concentric annular zones, which requires each zone to become progressively narrower from the center to the edge of the lens. There may be, for example, about 5 to 30 zones between the center and the edge of the lens. The surface profile within each zone is typically a smoothly varying function such as an arc, a parabola, or a line. At the outer periphery of each zone there is a discrete step in the vertical surface profile. The resulting surface structure can act as a circularly symmetric diffraction grating that disperses light into multiple diffraction orders, each diffraction order having a consecutive number, zero, one, two, three and so forth.

Diffractive IOLs lenses may be used for correcting presbyopia. In such an application, the lens can include one refractive surface and one diffractive surface. In practice, the light energy passing through a diffractive lens is typically concentrated into one, two, or three diffractive orders, while contributing an insignificant amount of light energy to other diffractive orders.

Existing designs for multifocal IOLs use either refractive optics, a combination refractive/diffractive design, or diffractive lenses that direct light into a single diffractive order. However, the fabrication of such IOLs can be time-consuming and expensive. Therefore, there is a need for improved ophthalmic lenses, particularly for improved diffractive IOLs that can be more readily fabricated.

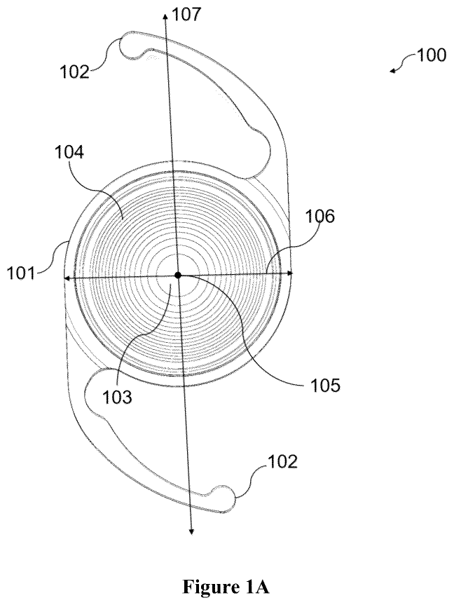

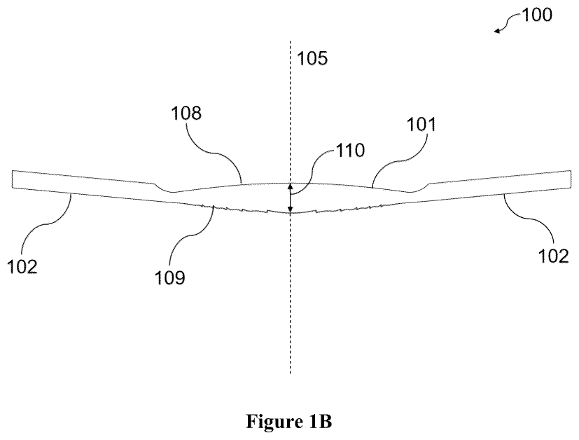

The present disclosure is directed to an intraocular lens (IOL), which provides an extended vision range. FIG. 1A shows a top view of a double-sided aspheric multifocal diffractive IOL 100, according to some embodiments of the present disclosure. FIG. 1B shows a cross-sectional view of the double-sided aspheric multifocal diffractive IOL 100, according to some embodiments of the present disclosure. IOL 100 can include a light transmissive circular disk-shaped lens body 101 with an optic diameter of 106 and a center thickness 110, as well as a pair of haptics 102 as flexible support for the IOL when implanted into patient's eye, with a total outer diameter 107. Lens body 101 can include an anterior surface 108, a posterior surface 109, and a central zone 103 and a plurality of diffraction elements 104 on the posterior surface 109. Lens body 101 can include an optical axis 105 extending transverse to anterior surface 108 and posterior surface 109. A skilled artisan in the art will appreciate that the optical axis 105 is a virtual axis for purposes of referring to the optical properties of IOL 100. The pair of haptics 102 can be extended outwardly from the lens body 101 for supporting the IOL 100 after implanted in the human eye. In some embodiments, haptics 102 of IOL 100 can hold the IOL in place in the capsular bag.

In some embodiments, lens body 101 can take the shape of biconvex shape. Other shapes of lens body 101 can include, but are not limited to, plano-convex, biconcave, plano-concave shape, or combinations of convex and concave shapes. In some embodiments, both anterior surface 108 and posterior surface 109 can feature an aspheric structure, providing a double-sided asphericity for IOL 100.

Diffractive element 104 can include diffractive rings or steps or also known as diffractive zones having a characteristic radial separation to produce constructive interference at characteristic foci on the optic area of the IOL. In some embodiments, diffractive elements 104 can include about 3 to about 30 diffractive rings/zones. In some embodiments, diffractive elements 104 can include about 5, 10, 15, 20, or 25 diffractive rings/zones. The IOL can contain diffractive elements on one of the surfaces or both surfaces of the lens. In some embodiments, the diffractive elements 104 can be placed on the posterior surface of the IOL. In some embodiments, the diffractive elements can be placed at the posterior surface, because there is less light scattering effect at the posterior surface than at the anterior surface. The plurality of diffractive elements 104 can include rings or zones extending concentrically with respect to the optical axis 105 through the central zone 103 over at least part of the posterior surface 109 of the lens body 101. The diffraction elements 104 can provide a focal point of far, intermediate, and/or near distance. In some embodiments, diffraction elements 104 are not limited to concentric circular or annular ring-shaped zones, but can include concentric elliptic or oval shaped zones.

In some embodiments, the optic diameter 106 of lens body 101 can be about 4 to about 8 mm, while the total outer diameter 107 of IOL 100 including the haptics 102 can be about 9 to about 18 mm. Lens body 101 can have a center thickness 110 of about 0.8 to about 1.2 mm. Although the embodiment in FIGS. 1A and 1B deals with a posterior chamber IOL, other ophthalmic lenses, including multifocal diffractive contact lenses or eye glass lenses, could also benefit from the same approach. When used for ophthalmic multifocal contact lenses and spectacle or eye glass lenses, haptics 102 are not provided.

The amount of correction that an ophthalmic lens provides is called optical power, and is expressed in Diopter (D). The optical power is calculated as the inverse of a focal distance f measured in meters, which can be a respective focal distance from the lens to a respective focal point for far, intermediate, or near vision. Lens body 101 in the double-sided aspheric shape of the present disclosure can provide a base optical power of about 10 to about 25 D. In some embodiments, lens body 101 can provide a base optical power of about 12, 14, 16, 18, 20, 22, or 24 D. The plurality of diffractive elements 104 can provide add power f₁=f₀+2.2 D and f₂=f₀+3.3 D.

IOLs can be made of flexible material which permits a reduction of their overall apparent girth by temporary deformation, facilitating their insertion through the cornea, thereby advantageously enabling the use of a corneal incision of concomitantly reduced size. In some embodiments, the lens body can include polypropylene, polycarbonate, polyethylene, acryl-butadiene styrene, polyamide, polychlorotrifluorocthylene, polytetrafluorocthylene, polyvinyl chloride, polyvinylidene fluoride, polyvinylchloride, polydimethylsiloxane, polyethylene terephthalate, ethylene tetrafluoroethylene, ethylene chlorotrifluoroethylene, perfluoroalkoxy, polymethylpentene, polymethylmethacrylate, polystyrene, polyetheretherketone, tetrafluoroethylene, polyurethane, poly(methyl methacrylate), poly (2-hydroxyethyl methacrylate), nylon, polyether block amide, silicone or a mixture thereof.

In some embodiments, the lens body can include a hydrophilic polymer made of monomers selected from the group consisting of: 2-acrylamido-2-methylpropane sulfonic acid, 2-hydroxyethyl methacrylate, N-vinylpyrrolidone, vinylbenzyltrimethyl ammonium salt, diethylaminoethyl methacrylate, dimethylaminoethyl acrylate, dimethylaminoethyl methacrylate, diethylaminoethyl acrylate, diethylaminomethyl methacrylate, tertiary butylaminoethyl acrylate, tertiary-butylaminoethyl methacrylate and dimethylaminopropylacrylamide, acrylic acid, methacrylic acid, styrenesulfonic acid and salts thereof, hydroxypropyl acrylate, vinylpyrrolidone, dimethylacrylamide, ethylene glycol monomethacrylate, ethylene glycol monoacrylate, ethylene glycol dimethacrylate, ethylene glycol diacrylate, triethylene glycol diacrylate and triethylene glycol methacrylate. In some embodiments, these hydrophilic monomers are surface grafted onto the polymeric matrix in the previous paragraph to make the lens body. In some embodiments, the IOL of the present disclosure can be made of polymeric compositions according to U.S. Pat. No. 10,494,458, which is incorporated herein by reference in its entirety.

The haptics of the IOL according to the present disclosure can be made of polymeric materials including, but not limited to polymethacrylate, polypropylene, polyethylene, polystyrene, and polyacrylate.

The surface of the IOL can include a spheric, aspheric, or toric element. Spheric surfaces can cause spherical aberration, which is a type of optical imperfection that can cause increased glare, and reduced overall quality of vision especially in low light and darkness. Aspheric lenses can correct spherical aberration. Aspherical IOL can provide improved contrast sensitivity, enhanced functional vision and superior night driving ability.

A toric element is typically used for astigmatic eye correction. Generally, astigmatism is an optical defect in which vision is blurred due to the ocular inability to focus a point object into a sharply focused image on the retina. This may be due to an irregular curvature of the cornea and/or lens. The refractive error of the astigmatic eye stems from a difference in degree of curvature, and therefore in degree of refraction, of the different meridians of the cornea and/or the crystalline lens, which causes the eye to have two focal points, one correspondent to each meridian. As used herein, a meridian includes one of two axes that subtend a curved surface, such as the prime meridian on the earth, for example. Meridians may be orthogonal. By way of example, the meridians of the earth may be any orthogonal line of longitude and any line of latitude that curve about the surface of the earth.

For example, in an astigmatic eye, an image may be clearly focused on the retina in the horizontal (sagittal) plane, but may be focused behind the retina in the vertical (tangential) plane. In the case where the astigmatism results only from the cornea, the two astigmatism meridians may be the two axes of the cornea. If the astigmatism results from the crystalline lens, the two astigmatism meridians may be the two axes of the crystalline lens. If the astigmatism results from a combination of the cornea and the crystalline lens, the two astigmatism meridians may be the respective axes of the combined lenses of the cornea and the crystalline lens.

An astigmatism arising from the cornea or crystalline lens, or the combination of the two lenses, may be corrected by a lens including a toric component. A toric surface resembles a section of the surface of a football, for which there are two regular radii of curvature, one smaller than another. These radii may be used to correct the defocus in the two meridians of the astigmatic eye. Thus, blurred vision caused by astigmatism may be corrected by corrective lenses or laser vision correction, such as glasses, hard contact lenses, contact lenses, and/or an IOL, providing a compensating optic specifically rotated around the optical axis.

In some embodiments, the IOL according to the present disclosure can provide far vision for viewing objects at distances ranging from about infinity to about 4 meters (m). In some embodiments, the IOL of the present disclosure can provide near vision for viewing objects at distances less than about 0.4 m. In some embodiments, the IOL of the present disclosure can provide intermediate vision for viewing objects at distances in a range of about 0.4 to about 1 m, about 2 m, about 3 m, or about 4 m. As a result, the IOL of the present disclosure can advantageously provide a degree of accommodation for different distance ranges, typically referred to as pseudo-accommodation. In some embodiments, when implanted into a patient's eye, the combined power of the eye's cornea and the near, intermediate, and far power of the IOL of the present disclosure can allow focusing light emanating from objects within a near, an intermediate, and a far distance range of the patient onto the retina. In some embodiments, the distance focus (f₀), intermediate focus (f₁), and near focus (f₂) provided by the IOL of the present disclosure can have the following ranges:

$$0 D \leq \frac{1}{{f}_{0}} \leq 55 D ,$$

$$D \leq \frac{1}{{f}_{1}} - \frac{1}{{f}_{0}} \leq 2.5 D , and$$

$$2 D \leq \frac{1}{{f}_{2}} - \frac{1}{{f}_{0}} \leq 5 D .$$

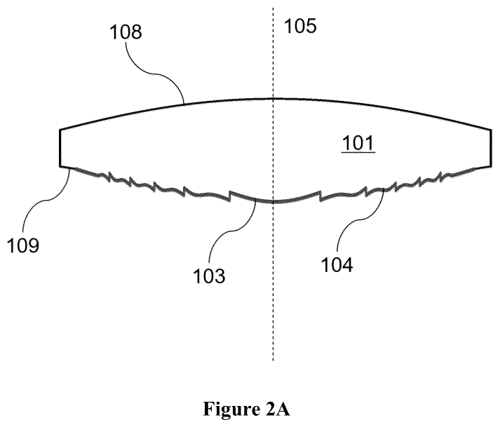

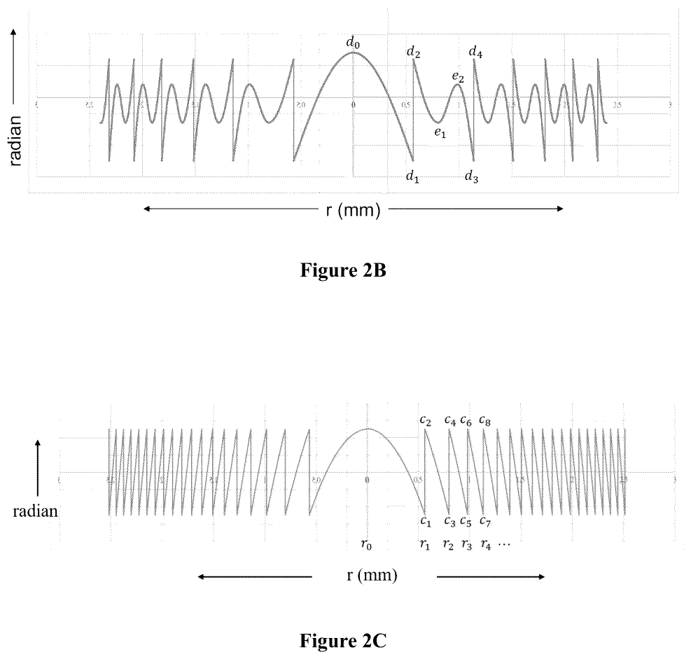

FIG. 2A shows a blow-up view of lens body 101, including anterior surface 108, posterior surface 109, optical axis 105, central zone 103 and the plurality of diffraction elements 104 generated on the posterior surface. The central zone 103 and diffraction elements 104 are further illustrated in FIG. 2B. The central zone begins from the 0^{th} Fresnel zone (from d₀ to d₁). The diffraction elements are configured as periodically structured smooth curve (from d₂ to d₃), each periodic structure of the diffraction elements contains two smooth turning points (e₁, e₂) in between two sharp turning points (d₂, d₃). FIG. 2C illustrates another embodiment of the present disclosure by smoothing out two smooth turning points (e₁, e₂) and their periodically corresponding turning points.

This diffractive structure embodied on the IOLs of the present disclosure can be designed using Equations (I) to (IV) as discussed below.

Pupil Function. A pupil function is a lens characteristic function that describes the physical effect of a lens by which it is possible to change the state of light made incident on the lens, and in specific terms, is represented by the product of the amplitude function A(r) and the exponential function of the phase function Φ₍ₙ₎(r) as noted in Equation (I) below.

$$T ( r ) = A ( r ) {e}^{i ( {Φ}_{( n )} ( r ) )} \qquad Equation ( I )$$

T(r): pupil function
A(r): amplitude function
Φ₍ₙ₎(r): phase function
n: natural number

Phase Function. A phase function is defined as the function that mathematically expresses the physical effect provided in a lens such as giving changes in the phase of incident light on a lens (position of wave peaks and valleys) using any method. The variable of the phase function is mainly expressed by position r in the radial direction from the center of the lens, and the phase of light made incident on the lens at the point of the position r undergoes a change by the phase function Φ₍ₙ₎(r) and is emitted from the lens. In specific terms, this is represented by an r-Φ coordinate system. In the present disclosure, phase is noted as Φ, and the unit is radians. One wavelength of light is represented as 2π radians, and a half wavelength as π radians, for example. A distribution of phase in the overall area in which the phase function is provided expressed in the same coordinate system is called a phase profile, or is simply called a profile or zone profile. With an r axis of Φ=0 as a reference line, this means that the light made incident at the point of Φ=0 is emitted without changing the phase. Also, for this reference line, when a positive value is used for Φ, this means that progress of the light is delayed by that phase amount, and when a negative value is used for Φ, this means that progress of the light is advanced by that phase amount. In an actual ophthalmic lens, a refracting surface for which a diffractive structure is not given corresponds to this reference line (surface). Light undergoes a phase change based on this phase function and is emitted from the lens.

Amplitude Function. An amplitude function is the function expressed by A(r) in Equation (I) noted above. In the present disclosure, this is defined as a function that represents the change in the light transmission amount when passing through a lens. The variable of the amplitude function is represented as position r in the radial direction from the center of the lens, and represents the transmission rate of the lens at the point of position r. Also, the amplitude function is in a range of 0 or greater and 1 or less, which means that light is not transmitted at the point of A(r)=0, and that incident light is transmitted as it is without loss at the point of A(r)=1.

Zone. In the present disclosure, a zone is used as the minimum unit in a diffractive structure, element, or diffraction grating provided in a lens.

The height profile of the diffractive structure (Z_{diff}) on the IOL can be calculated based on Equation (II) below.

$${Z}_{diff} ( r ) = {Φ}_{( n )} ( r ) \times \frac{λ}{{n}_{1} - {n}_{0}} \qquad Equation ( II )$$

Z_{diff}(r): height profile of the diffractive structure
Φ₍ₙ₎(r): phase function
λ: design wavelength
n₁: refractive index of the lens material
n₀: refractive index of the medium covering the lens

The radius of a particular diffractive zone (rₙ) can be calculated based on Equation (III) below.

$${r}_{n} = \sqrt{2 \times λ \times n \times f} \qquad Equation ( III )$$

rₙ: radius of the n^{th} zone
λ: design wavelength
f: reciprocal of add power

Phase function (Φ₍ₙ₎(r)) can be calculated via Equation (IV) below.

$${Φ}_{( n )} ( r ) = A \times sin ( ( B \times \frac{r - {r}_{n}}{{r}_{n + 1} - {r}_{n}} + C ) \times π ) + D \qquad Equation ( IV )$$

Φ₍ₙ₎(r): phase function
r: is the radial distance from a center of lens
rₙ: radius of the n^{th} zone
rₙ₊₁: radius of the (n+1)^{th} zone
   wherein A, B, C and D, are the light distribution parameters. A is the amplitude: B is the period as 2π; C is the phase shift, if it is +C it shifts left, if the phase shift is −C, it shifts right; D is the vertical shift, if it is +D, the function moves up, if it is −D, then the function moves down.

The double-sided aspheric structure (anterior and posterior of the optic area of the IOL) is for the correction of the spherical aberration of the lens. The height profile of the aspheric base structure (Zₐₛₚ) of the lens can be calculated according to the following Equation (V):

$${Z}_{asp} ( r ) = \frac{c {r}^{2}}{1 + \sqrt{1 - ( 1 + k ) {c}^{2} {r}^{2}}} + {\sum }_{i = 2}^{n} {A}_{i} {r}^{2 i} \qquad Equation ( V )$$

Zₐₛₚ: is the height profile of the aspheric structure
r: is the radial distance from a center of lens
k: is the conic constant
c: is the curvature
Aᵢ: is the high order aspheric coefficient

When both aspheric and diffractive structures are placed onto the same surface (anterior surface and/or posterior surface of the IOL), according to some embodiments of the present disclosure, the height profile of the combination structure (Zₜₒₜₐₗ) will be the summation of the height profile of the aspheric structure (Zₐₛₚ) and the height profile of the diffractive structure (Z_{diff}), as calculated according to the below Equation (VI).

$${Z}_{total} ( r ) = {Z}_{asp} ( r ) + {Z}_{diff} ( r ) \qquad Equation ( VI )$$

Z_{diff}: height profile of the diffractive structure
Z_{aspheric}: height profile of the aspheric structure
Zₜₒₜₐₗ: height profile of the combination structure, i.e. the lens body

In some embodiments, the above-described lens can be contact lens or IOL. In some embodiments, the IOL can be intracorneal IOL, anterior chamber IOL or posterior chamber IOL. In some embodiments, the IOL can be posterior chamber IOL. While the haptic arms are illustrated in the embodiment, any suitable haptics fixation structure for the capsular bag or the ciliary sulcus compatible with posterior chamber implantation can also be used in a posterior chamber IOL.

A way of estimating the optical priority of an intraocular lens comprises determining experimentally its modulation transfer function (MTF). The MTF of an optical system can be measured according to Annex C of ISO 11979-2, which reflects the proportion of the contrast which is transmitted through the optical system for a determined spatial frequency of a test pattern, which frequency is defined as “cycles/mm” or “LP/mm”, in which “LP” indicates “line pairs.” Generally, the contrast decreases with an increase in spatial frequency.

All publications, patents, and patent applications mentioned in the present disclosure are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, it will be understood that modifications and variations are encompassed within the spirit and scope of the instant disclosure. The preferred methods and materials are now described.

Presented below are examples discussing different embodiments of the IOLs contemplated for the discussed applications. The following examples are provided to further illustrate the embodiments of the present disclosure, but are not intended to limit the scope of the disclosure. While they are typical of those that might be used, other procedures, methodologies, or techniques known to those skilled in the art may alternatively be used.

### EXAMPLES

### Example 1

### MTF and Height Profile of the IOL According to the First Embodiment of the Present Disclosure

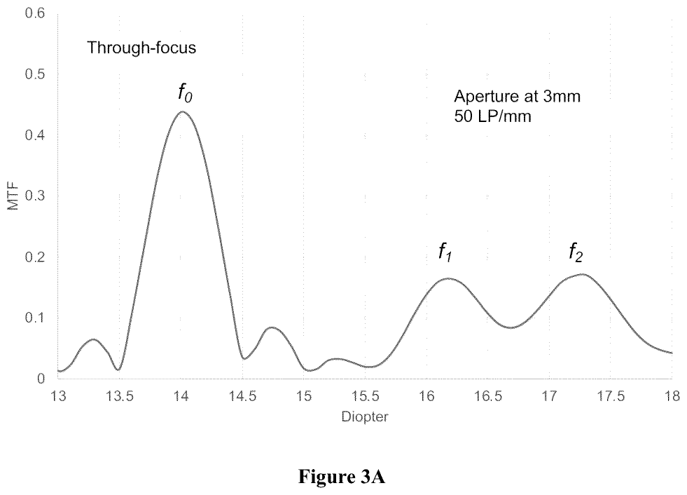

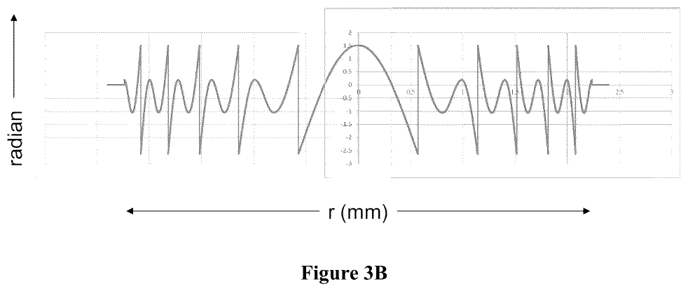

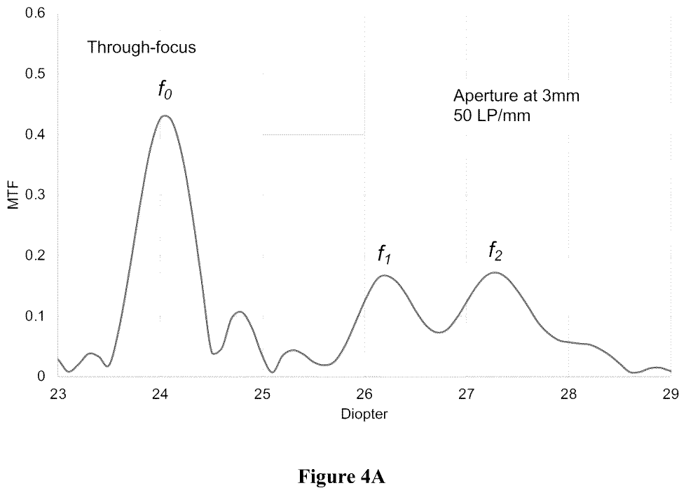

From Equation (IV), by varying parameters A, B, C and D, and controlling the distance focus (f₀), the intermediate focus (f₁), and the near focus (f₂), the optical performance (modulation transfer function, MTF) at a 3 mm aperture and at a resolution measurement of 50 line pairs per millimeter (LP/mm) is shown in FIG. 3A. The parameters of A, B, C and D are varied according to Table 1 below.

TABLE 1
Variation of parameters A-D in a first
embodiment of the present disclosure.

| | A | B | C | D |
|---|---|---|---|---|
| Ring 1 | 0.6 | 0.5 | 1 | −0.42 |
| Ring 2 | 0.41 | 0.5 | 0.5 | 0.24 |
| Ring 3 | −0.1 | 1 | 0.5 | −0.07 |
| Ring 4 | 0.45 | 0.5 | 1 | −0.42 |
| Ring 5 | 0.41 | 0.5 | 0.5 | 0.24 |
| Ring 6 | −0.1 | 1 | 0.5 | −0.07 |
| Ring 7 | 0.45 | 0.5 | 1 | −0.42 |
| Ring 8 | 0.41 | 0.5 | 0.5 | 0.24 |
| Ring 9 | −0.1 | 1 | 0.5 | −0.07 |
| Ring 10 | 0.45 | 0.5 | 1 | −0.42 |
| Ring 11 | 0.41 | 0.5 | 0.5 | 0.24 |
| Ring 12 | −0.1 | 1 | 0.5 | −0.07 |
| Ring 13 | 0.45 | 0.5 | 1 | −0.42 |
| Ring 14 | 0.41 | 0.5 | 0.5 | 0.24 |
| Ring 15 | −0.1 | 1 | 0.5 | −0.07 |

The curve in FIG. 3A shows three peaks corresponding to a distance focus at about 14.0 D, an intermediate focus at about 16.2 D, and a near focus at about 17.3 D, respectively.

The Zₜₒₜₐₗ (r) height profile of the aspheric and diffractive combination structure is shown in FIG. 3B. The height is depicted at μm scale along the vertical axis. The optical axis, running through the center of the lens body, is assumed to be at a radial position r=0, whereas the radial distance r measured in outward direction from the optical axis is expressed in mm along the vertical axis.

### Example 2

### MTF and Height Profile of the IOL According to the Second Embodiment of the Present Disclosure

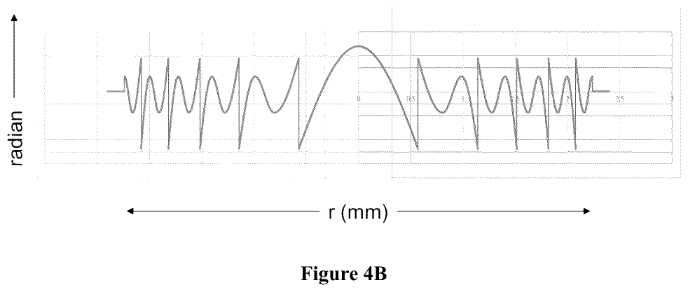

From Equation (IV), by varying parameters A, B, C and D, and controlling the f₀, f₁ and f₂, the optical performance (MTF) at a 3 mm aperture and at a resolution measurement of 50 line pairs per millimeter (LP/mm) is shown in FIG. 4A. The parameters of A, B, C and D are varied according to Table 2 below. The Zₜₒₜₐₗ (r) height profile of the aspheric and diffractive combination structure is shown in FIG. 4B.

TABLE 2
Variation of parameters A-D in a second
embodiment of the present disclosure.

| | A | B | C | D |
|---|---|---|---|---|
| Ring 1 | 0.68 | 0.5 | 1 | −0.38 |
| Ring 2 | 0.36 | 0.5 | 0.5 | 0.22 |
| Ring 3 | −0.12 | 1 | 0.5 | −0.02 |
| Ring 4 | 0.48 | 0.5 | 1 | −0.38 |
| Ring 5 | 0.36 | 0.5 | 0.5 | 0.22 |
| Ring 6 | −0.12 | 1 | 0.5 | −0.02 |
| Ring 7 | 0.48 | 0.5 | 1 | −0.38 |
| Ring 8 | 0.36 | 0.5 | 0.5 | 0.22 |
| Ring 9 | −0.12 | 1 | 0.5 | −0.02 |
| Ring 10 | 0.48 | 0.5 | 1 | −0.38 |
| Ring 11 | 0.36 | 0.5 | 0.5 | 0.22 |
| Ring 12 | −0.12 | 1 | 0.5 | −0.02 |
| Ring 13 | 0.48 | 0.5 | 1 | −0.38 |
| Ring 14 | 0.36 | 0.5 | 0.5 | 0.22 |
| Ring 15 | −0.12 | 1 | 0.5 | −0.02 |

The curve in FIG. 4A shows three peaks corresponding to a distance focus at about 24.0 D, an intermediate focus at about 26.2 D, and a near focus at about 27.3 D, respectively.

### Example 3

### MTF and Height Profile of the IOL According to the Third Embodiment of the Present Disclosure

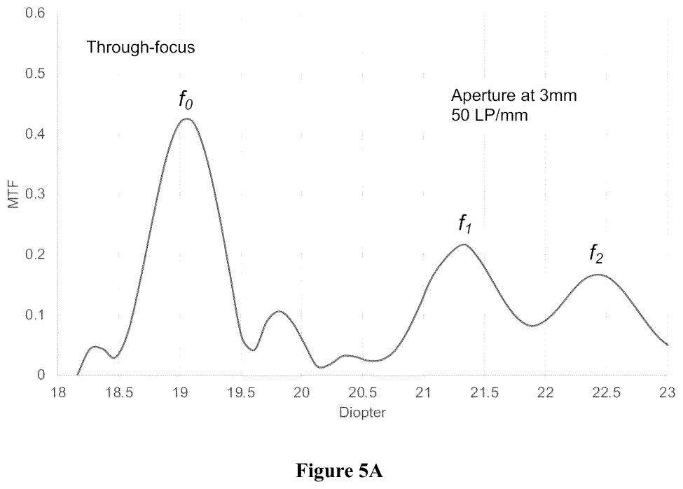

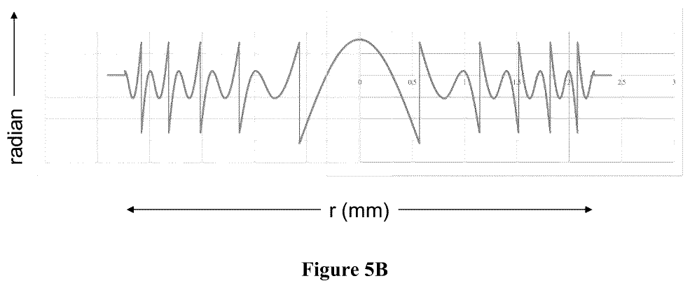

From Equation (IV), by varying parameters A, B, C and D, and controlling the f₀, f₁ and f₂, the optical performance (MTF) at a 3 mm aperture and at a resolution measurement of 50 line pairs per millimeter (LP/mm) is shown in FIG. 5A. The parameters of A, B, C and D are varied according to the Table 3 below. The Zₜₒₜₐₗ(r) height profile of the aspheric and diffractive combination structure is shown in FIG. 5B.

TABLE 3
Variation of parameters A-D in a third
embodiment of the present disclosure.

| | A | B | C | D |
|---|---|---|---|---|
| Ring 1 | 0.76 | 0.5 | 1 | −0.5 |
| Ring 2 | 0.41 | 0.5 | 0.5 | 0.24 |
| Ring 3 | −0.1 | 1 | 0.5 | −0.07 |
| Ring 4 | 0.45 | 0.5 | 1 | −0.42 |
| Ring 5 | 0.41 | 0.5 | 0.5 | 0.24 |
| Ring 6 | −0.1 | 1 | 0.5 | −0.07 |
| Ring 7 | 0.45 | 0.5 | 1 | −0.42 |
| Ring 8 | 0.41 | 0.5 | 0.5 | 0.24 |
| Ring 9 | −0.1 | 1 | 0.5 | −0.07 |
| Ring 10 | 0.45 | 0.5 | 1 | −0.42 |
| Ring 11 | 0.41 | 0.5 | 0.5 | 0.24 |
| Ring 12 | −0.1 | 1 | 0.5 | −0.07 |
| Ring 13 | 0.45 | 0.5 | 1 | −0.42 |
| Ring 14 | 0.41 | 0.5 | 0.5 | 0.24 |
| Ring 15 | −0.1 | 1 | 0.5 | −0.07 |

The curve in FIG. 5A shows three peaks corresponding to a distance focus at about 19.1 D, an intermediate focus at about 21.2 D, and a near focus at about 22.3 D, respectively.

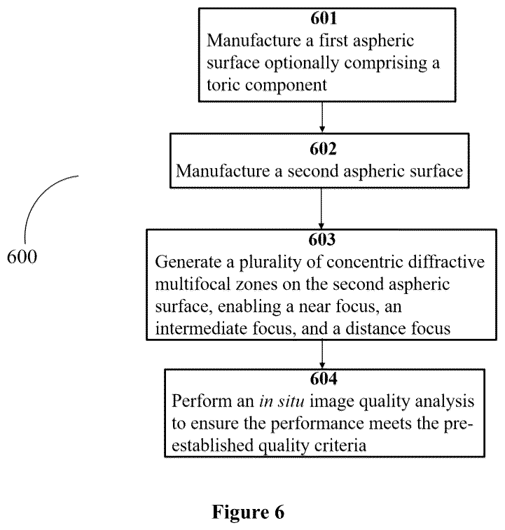

FIG. 6 is a flowchart 600 illustrating the design and manufacture of the double-sided aspheric multifocal diffractive IOL, according to some embodiments of the present disclosure. Step 601 manufactures a first aspheric surface optionally including a toric component. Step 602 manufactures a second aspheric surface. Step 603 generates a plurality of concentric diffractive multifocal zones on the second aspheric surface to produce a near focus, an intermediate focus, and a distance focus. Step 604 performs an in situ image quality analysis of the double-sided aspheric diffractive multifocal lens on an eye to measure the through focus MTF using the TRIOPTICS OptiSpheric® IOL PRO 2 up toe the pre-established ISO Model Eye 2 performance criteria.

While the disclosure has been particularly shown and described with reference to specific embodiments, it should be understood by those having skill in the art that various changes in form and detail may be made therein without departing from the spirit and scope of the present disclosure as disclosed herein.

All references referred to in the present disclosure are hereby incorporated by reference in their entirety. Various embodiments of the present disclosure may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

The embodiments of the disclosure described above are intended to be merely exemplary; numerous variations and modifications will be apparent to those skilled in the art. All such variations and modifications are intended to be within the scope of the present disclosure as defined in any appended claims.

1-20. (canceled)

21. A diffractive trifocal lens comprising a lens body, the lens body comprising:
a first aspheric surface; and
a second aspheric surface comprising a central zone and a plurality of diffractive elements comprising concentric annular zones extending in a radial direction, each concentric annular zone having a periodically structured curve comprising two smooth turning points between two sharp turning points, thereby producing a near focus (f₂), an intermediate focus (f₁), and a distance focus (f₀),
wherein a modulation transfer function (MTF) of the diffractive trifocal lens comprises a first peak at the intermediate focus (f₁) and a second peak at the near focus (f₂), and
wherein a most minimum value of the MTF between the first peak and the second peak is about 50% of a most maximum value of the MTF at the first peak, thereby improving vision contrast between the intermediate focus (f₁) and the near focus (f₂).

22. The diffractive trifocal lens of claim 21, wherein a slope of the periodically structured curve changes sign at each of the two smooth turning points in each of the concentric annular zones.

23. The diffractive trifocal lens of claim 21, wherein:
an optical power of the distance focus (f₀) is greater than or equal to 0 diopters and less than or equal to 55 diopters;
an optical power of the intermediate focus (f₁) is greater than the optical power of the distance focus (f₀) by a first difference in optical power that is greater than or equal to 1 diopter and less than or equal to 2.5 diopters; and
an optical power of the near focus (f₂) is greater than the optical power of the distance focus (f₀) by a second difference in optical power that is greater than or equal to 2 diopters and less than or equal to 5 diopters.

24. The diffractive trifocal lens of claim 21, wherein the diffractive trifocal lens produces the distance focus for viewing objects at a distance greater than about 4 m.

25. The diffractive trifocal lens of claim 21, wherein the diffractive trifocal lens produces the near focus for viewing objects at distance less than about 0.4 m.

26. The diffractive trifocal lens of claim 21, wherein the diffractive trifocal lens produces the intermediate focus for viewing objects at a distance between about 0.4 m to about 4 m.

27. The diffractive trifocal lens of claim 21, wherein the first aspheric surface is an anterior surface of the lens body, and the second aspheric surface is a posterior surface of the lens body.

28. The diffractive trifocal lens of claim 21, wherein the first aspheric surface comprises a toric component.

29. The diffractive trifocal lens of claim 21, wherein the lens body has a biconvex shape.

30. The diffractive trifocal lens of claim 21, wherein the second aspheric surface comprises between 3 and about 30 diffractive elements.

31. The diffractive trifocal lens of claim 21, comprising a pair of haptics extended outwardly from the lens body.

32. The diffractive trifocal lens of claim 21, wherein the diffractive trifocal lens is an intraocular lens (IOL) sized for insertion into a human eye.

33. The diffractive trifocal lens of claim 32, wherein the IOL is sized for insertion into a posterior chamber of the human eye.

34. The diffractive trifocal lens of claim 33, wherein the IOL is configured to be implanted into a capsular bag of the posterior chamber of the human eye.

35. A method of treating an ophthalmic disease or disorder in a subject, the method comprising implanting into an eye of the subject a diffractive trifocal lens comprising a lens body, the lens body comprising:
a first aspheric surface; and
a second aspheric surface comprising a central zone and a plurality of diffractive elements comprising concentric annular zones extending in a radial direction, each concentric annular zone having a periodically structured curve comprising two smooth turning points between two sharp turning points, thereby producing a near focus (f₂), an intermediate focus (f₁), and a distance focus (f₀),
wherein a modulation transfer function (MTF) of the diffractive trifocal lens comprises a first peak at the intermediate focus (f₁) and a second peak at the near focus (f₂), and
wherein a most minimum value of the MTF between the first peak and the second peak is about 50% of a most maximum value of the MTF at the first peak, thereby improving vision contrast between the intermediate focus (f₁) and the near focus (f₂).

36. The method of claim 35, wherein the ophthalmic disease or disorder is selected from the group consisting of cataract and presbyopia.

37. The method of claim 35, wherein the diffractive trifocal lens is an intraocular lens (IOL) sized for insertion into the subject's eye.

38. The method of claim 37, wherein the IOL is implanted into a capsular bag of a posterior chamber of the subject's eye.

39. A method of manufacturing a diffractive trifocal lens, the method comprising:
(a) manufacturing a first aspheric surface optionally comprising a toric component on a side of a lens body;
(b) manufacturing a second aspheric surface on a side of the lens body opposite the side having the first aspheric surface; and
(c) generating, on the second aspheric surface, a central zone and a plurality of diffractive elements comprising a plurality of concentric annular zones extending in a radial direction, each concentric annular zone having a periodically structured curve comprising two smooth turning points between two sharp turning points, thereby producing a near focus (f₂), an intermediate focus (f₁), and a distance focus (f₀),
wherein a modulation transfer function (MTF) of the diffractive trifocal lens comprises a first peak at the intermediate focus (f₁) and a second peak at the near focus (f₂), and
wherein a most minimum value of the MTF between the first peak and the second peak is about 50% of a most maximum value of the MTF at the first peak, thereby improving vision contrast between the intermediate focus (f₁) and the near focus (f₂).

40. The method of claim 39, comprising:
performing an in situ image quality analysis to assess optical performance of the diffractive trifocal lens by measuring the modulation transfer function (MTF) of the trifocal lens using pre-established quality criteria.
